(19) United States
(12) Patent Application Publication
(10) Pub. No.: US 2012/0040394 A1
(43) Pub. Date: Feb. 16, 2012

(54) MICROTUMOURS

(76) Inventors: Paul Raju, Oxfordshire (GB); Zhanfeng Cui, Oxfordshire (GB)

(21) Appl. No.: 13/121,351

(22) PCT Filed: Sep. 28, 2009
(86) PCT No.: PCT/GB2009/051269

§ 371 Date: Nov. 7, 2011

(30) Foreign Application Priority Data
Sep. 29, 2008 (GB) 0817783.4

(51) Int. Cl. C12Q 1/18; C12Q 1/02; C07K 16/18; C12N 5/09

(52) U.S. Cl. 435/32; 435/395; 435/29; 530/389.7; 435/366; 435/325

(57) ABSTRACT

A method of producing an in vitro microtumour comprising: seeding a colorectal neoplastic cell into a three dimensional scaffold comprising polysaccharide co-polymer; providing said cell with a culture medium that supports the growth thereof; and incubating said cell in said scaffold for a time sufficient for microtumors to form, wherein said polysaccharide copolymer comprises glutaronate and mannuronate.

The present invention relates to an in vitro method of producing a microtumor. More particularly the invention relates to an in vitro of method of producing a colorectal microtumor and screening methods using said microtumors.

### BACKGROUND

Neo-plastic monolayers are poor models for studying cancer biology and screening drugs because of their limited predictive power of in vivo physiology. While monolayer cultures are advantageous for many applications, they are less useful when studying cancer biology in vitro. The monolayer system is acknowledged to be quite different from the environment of the in vivo tumor, since they inherently lack the ability to simulate cell-cell and cell-matrix interactions in three dimensions; in particular, the extracellular matrix which plays a crucial role in tumour progression is absent. Moreover, 2D cultures provide little information on the kinetics of the growth factors important for cancer development

For this reason, 3D tumor spheroids have gained prominence for the testing of putative drugs and in the study of proliferation, metabolism, metastasis and angiogenesis. Tumour spheroids are cancer cell aggregates, which possess a natural 3D configuration. Spheroids are an artificial phenomenon where mono-dispersed cells from cancerous or normal-healthy tissue origin can be coaxed into forming a self-adhering ball of cells.

Both normal and cancerous cells can be coaxed into forming a multi-celled spheroid. For example: embryonic stem cells forming embryoid bodies (Kurosawa, Imamura et al. J Biosci Bioeng 96(4): 409-11, 2003), hepatocytes into liver spheroids (Wu, Friend et al. Cell Transplant 8(3): 233-46, 1999), glial & neuronal cells into brain spheroids (Chatterjee and Noldner, J Neural Transm Suppl 44: 47-60 1994) and cancerous cells into tumor spheroids (Nicholson, Bibby et al. Eur J Cancer 33(8): 1291-8 1997). Processes for the formation of spheroids are described in U.S. Pat. No. 7,052,720, U.S. Pat. No. 5,624,839 and Durand et al Cancer Res., 33:213-219, 1973. Conventional methods employed for generating multi-cellular spheroids from single cell suspensions are liquid overlay technique, spinner flask method, gyratory rotation systems (Santini and Rainaldi Pathobiology 67(3): 148-57 1999) or hanging drop (Kurosawa, Imamura et al. J Biosci Bioeng 96(4): 409-11 2003). The common principle behind these methods is inhibition of surface-cell attachment; this keeps the cells in suspension and creates a drive for self adherence resulting in a multi-cellular ball that propagates.

In addition to their 3D aspect, tumour spheroids also exhibit some basic properties of their naturally occurring (in vivo tumours) counterparts, such as: anatomical regions of cell proliferation, quiescence and necrosis (Freyer and Sutherland 1986; Sutherland 1988; Mellor, Ferguson et al. 2005); tissue fidelity in regards to function, for example, cancer cell spheroids of colorectal cancer cells and thyroid produce copious amounts of carcinoembryonic antigen and thyroid hormone respectively, such fidelity is generally not seen in their monolayer counterparts (Mueller-Klieser 1987); response to cancer therapy, when compared to monolayers, tumour spheroids are more resistant to radiation due to their hypoxic (quiescent) regions within the spheroid (Franko 1985).

In spite of their similarities to actual tumours, tumour spheroids fall short of an ideal tumour for cancer research. Absent from tumour spheroids, but present in actual tumours, are infiltrating leukocytes (inflammatory cells), blood vessels (characteristic of metastatic tumours) and an interacting extracellular milieu, which seems to play a pivotal role in tumorgenesis and progression (Dvorak 1986; Dvorak, Nagy et al. 1991). In addition, the micro-environment around tumours is more acidic (pH 6.2-6.9) than compared to normal healthy cells (pH 7.3-7.4), (Gillies, Raghunand et al. 2002). The reversal of this pH gradient occurs early in tumour formation through oncogene-stimulation of the cell membrane Na+/H+ exchanger (Kaplan and Boron 1994). The subsequent alkalinization of the cell/tumour's interior induces cell proliferation independent of external control (serum and anchorage factors) which results in a mass of disorganized and dense tissue. A plethora of research finds a positive correlation between progressive acidity around a tumours micro-environment and its metastatic potential (Montcourrier, Mangeat et al. 1994; Montcourrier, Silver et al. 1997; Parkins, Stratford et al. 1997; Xu, Fukumura et al. 2002).

In addition, investigators have suffered low cell viabilities with spheroids when compared to their 2D counterparts, due to diffusion limitations in static cultures. Moreover it has been suggested that extra-cellular milieu may play a crucial role in tumor pathogenesis; a microenvironment of 3D scaffolds (matrigel) preconditioned with human embryonic stem cells has been shown to potentially reprogram melanoma tumor cells back to normal cell phenotype (Postovit, Seftor et al. 2006).

Accordingly, there remains a need for an in vitro tumor model which more accurately reflects the in vivo 3D structure of a tumor.

### BRIEF SUMMARY OF THE DISCLOSURE

According to a first aspect of the invention, there is provided a method of producing an in vitro microtumour comprising:
i. seeding a colorectal neoplastic cell into a three dimensional scaffold comprising polysaccharide co-polymer;
ii. providing said cell with a culture medium that supports the growth thereof; and
iii. incubating said cell in said scaffold for a time sufficient for microtumors to form,
   wherein said polysaccharide co-polymer comprises glutaronate and mannuronate.

Preferably, said cell is a colorectal adenocarcinoma cell. Preferably said cell comprises a CHK2 mutation. More preferably said cell is a colon cell. Still more preferably said cell is a DLD1 cell.

Preferably said culture medium is provided as a static culture. Alternatively, said culture medium is provided as a perfused culture.

Preferably said polysaccharide is an alginate or alginic acid.

In a further aspect the invention provides a method of screening a compound to identify agents useful for the treatment of cancer, comprising:
i) exposing a microtumor to a test compound; and
ii) determining the effect of the compound on the microtumor
   wherein said microtumor is produced by seeding a colorectal neoplastic cell into a three dimensional scaffold which comprises a polysaccharide co-polymer comprising glutaronate and mannuronate.

Preferably said microtumor is within said three dimensional scaffold.

Preferably said cell is a colorectal adenocarcinoma cell. Preferably said cell comprises a CHK2 mutation. Preferably said cell is a colon cell. More preferably said cell is a DLD1 cell.

Preferably the method comprises determining the cytotoxic effect of the test compound on the microtumor. Preferably the cytotoxic effect of the test compound on the microtumor is determined using a live/dead cytotoxicity assay.

Alternatively the method comprises determining the effect of the test compound on acidic compartments of the microtumor. Preferably the effect of the test compound on acidic compartments of the microtumor is determined by staining of the microtumor with acridine orange.

Alternatively, the method comprises determining the effect of the test compound on endogenous fluorescence of the microtumor. Preferably the effect of the test compound on endogenous florescence is determined by the visualisation of fluroproteins or NAD(P)H by NIR-MPM.

Alternatively, the method comprises determining the effect of the test compound on the cell cycle. Preferably the effect of the test compound on the cell cycle is determined by flow cytometry.

Alternatively the method comprises determining the cytostatic effect of the test compound on the microtumor.

Preferably said polysaccharide co-polymer comprising glutaronate and mannuronate is alginate or algenic acid.

In a further aspect the invention provides a process for preparing a pharmaceutical composition for treating a colorectal cancer comprising:

(a) screening a plurality of compounds using a microtumor produced by seeding a colorectal neoplastic cell into a three dimensional scaffold comprising glutaronate and mannuronate, to determine the effect of the compound on the microtumor;
(b) selecting from the plurality a compound having a cytotoxic or cytostatic action against said microtumor;
(c) synthesising the selected compound; and
(d) incorporating the synthesized compound into a pharmaceutical composition.

In a further aspect the invention provides use of a microtumor produced by seeding a colorectal neoplastic cell into a three dimensional scaffold comprising glutaronate and mannuronate to identify an agent useful for the treatment of cancer.

In a further aspect the invention provides a microtumor produced in accordance with the method described herein.

In a further aspect the invention provides a three dimensional alginate scaffold comprising a colorectal neoplastic cell aggregate which is resistant to trypsin digestion.

In a further aspect the invention provides a method of producing an in vitro microtumour as herein described.

In a further aspect the invention provides a method of screening a compound to identify agents useful for the treatment of cancer as herein described.

In a further aspect the invention a process for preparing a pharmaceutical composition for treating a colorectal cancer as herein described.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

Embodiments of the present invention will now be described hereinafter, by way of example only, with reference to the accompanying drawings in which:

FIG. 1 illustrates the spontaneous generation of micro-tumors (MT) in alginate scaffolds;

FIG. 2 provides a graphical comparison of the number of microtumors in static and perfused conditions;

FIG. 3a illustrates the nuclear and acidic components within a microtumor.

FIG. 3b provides a comprehensive map of nuclear and acidic components within a microtumor.

FIG. 3c illustrates the Acidic components distributed across a MT at various depths from the surface.

FIG. 3d illustrates the relative acidity of acidic components distributed across a microtumor.

FIG. 3e illustrates the 3D constructs of microtumors grown in alginate scaffolds;

FIG. 4 outlines quiescent (Q), proliferative (P) and necrotic (N) regions within the microtumor;

FIG. 5 illustrates the sensitivity of DLD1 and NCl/Adr cancer cells to the drug paclitaxel as a function of cellular acidity.

### DETAILED DESCRIPTION

Tissues, whether they be normal or cancerous, are closer to their native configuration when generated in a 3D, rather than a 2D, culture environment. Therefore biological measurements in artificial 3D scaffolds are more predictive of in vivo expectations.

The present inventors have surprisingly demonstrated that a colorectal cancer cell line, seeded in alginate scaffold as single cells, spontaneously forms microtumors. The inventors have demonstrated that microtumors progressively increase in number and mass over time, in both static and perfusion based culture systems.

Unlike other cell lines seeded in alginate scaffolds, which grow as mono dispersed cells, DLD1 colorectal cells spontaneously propagated as microtumors.

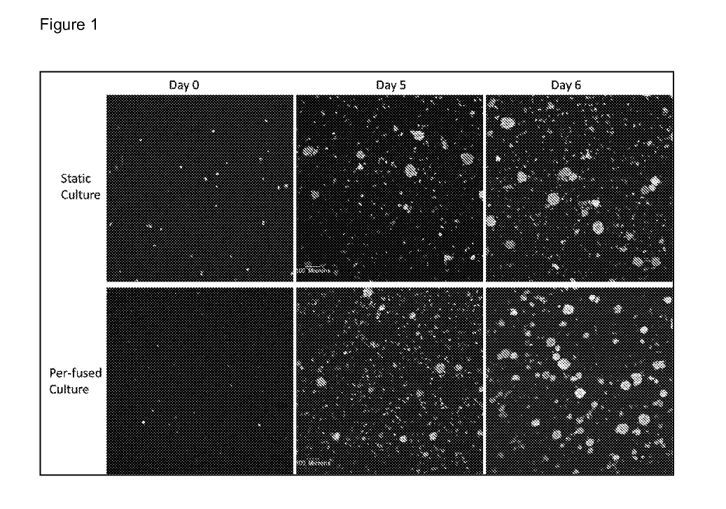

As shown in FIGS. 1a and b, more microtumors formed over a 6 day period in per-fused conditions than in static culture. In perfused conditions bioreactors provide cells/tissues with homeostatic conditions (optimal pH/temperature, nutrient supply and waste removal) for optimal growth to occur compared to static conditions.

Construction of artificial microtumors in a 3D scaffold has an immense potential not only for drug testing but in furthering the understanding of tumor biology.

The invention provides a 3D model in which infiltrating leukocytes may be incorporated into a tumor model. The invention provides a 3D model in which blood vessel formation is induced to give malignant properties. The invention provides a 3D model in which platform monitoring of extracellular pH is available, providing a potential means of assessing the effectiveness of a test compound. The invention provides a 3D model for testing the effect of conditioning of extracellular matrix on tumor formation, growth and metastasis. The invention provides a 3D model for screening test compounds for treating malignant tumors.

### Microtumors

Microtumors formed according to the present invention are distinct from spheroids. Spheroids are cell aggregates grown in suspension, for example in soft agar or methylcellulose, as opposed to a monolayer. It is usual for spheroids to stop enlarging at a diameter of a few millimetres as the spheroid will reach a steady state wherein the cell proliferation is balanced by cell death. The cell aggregate structure of the spheroid is susceptible to both mechanical and enzymatic dissociation. In contrast, the microtumours of the present invention are not amenable to mechanical or enzymatic dissociation, e.g. trypsin or lipase treatment, into single cells. For example, colorectal tumors extracted from patients required extensive enzymatic digestion in order to obtain single cell suspensions (Dierssen, de Miranda et al. BMC Cancer 6: 233. 2006) whereas DLD1 spheroids generated by conventional methods did not (Nicholson, Bibby et al. Eur J Cancer 33(8): 1291-8. 1997). A survey of literature indicates that a variety of spheroids are easily disassembled into single cells by tyrpsin digestion (Khaitan, Chandna et al. J Transl Med 4: 12. 2006, Sharma, Verma et al. Biotechnology Letters 29(2): 5. 2006, Burleson, Boente et al. J Transl Med 4: 6. 2006, Landry, Lord et al. Cancer Research 42: 6. 1982 and Freyer and Sutherland Cancer Res 46(7): 3504-12. 1986).

This inability to dissociate indicates that microtumors of the present application are distinct from spheroids as they possess a more rigid structure than cell aggregates, making them an ideal in vitro tumour model.

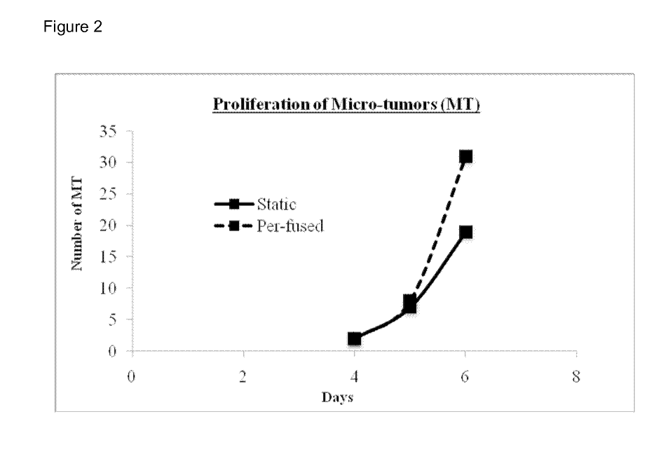

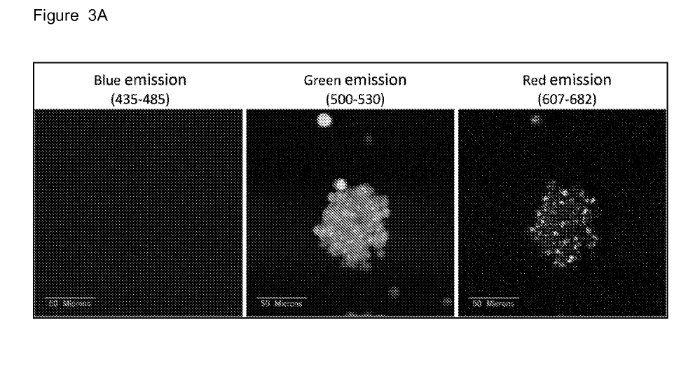

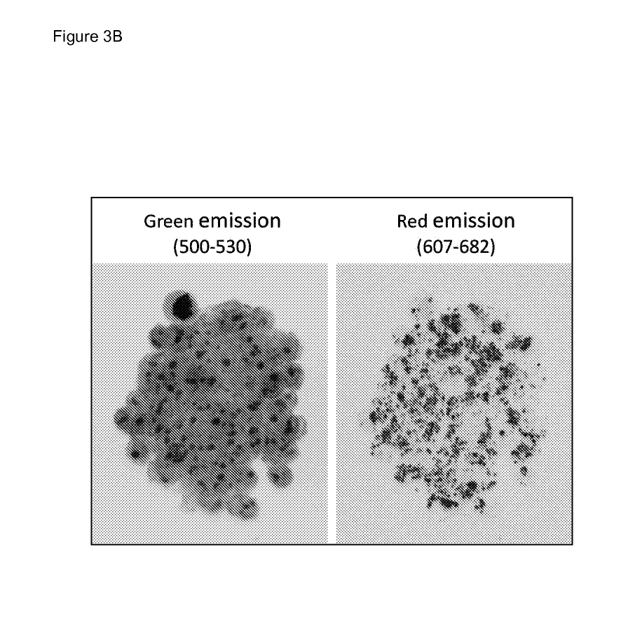

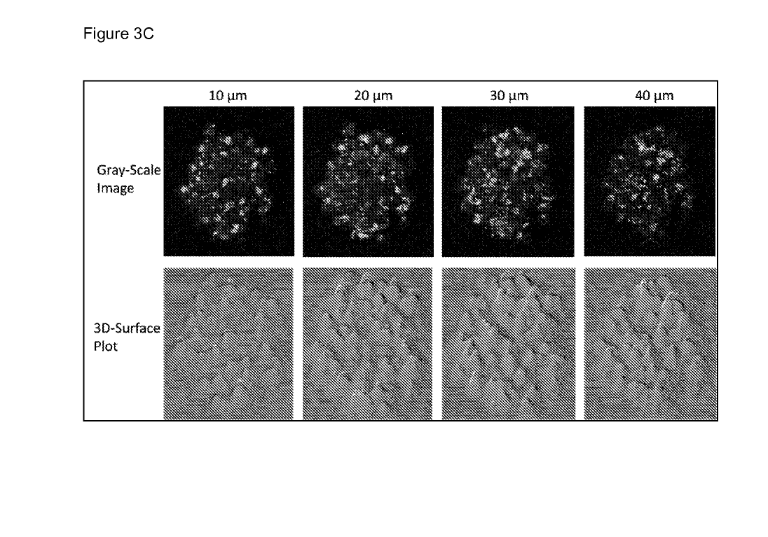

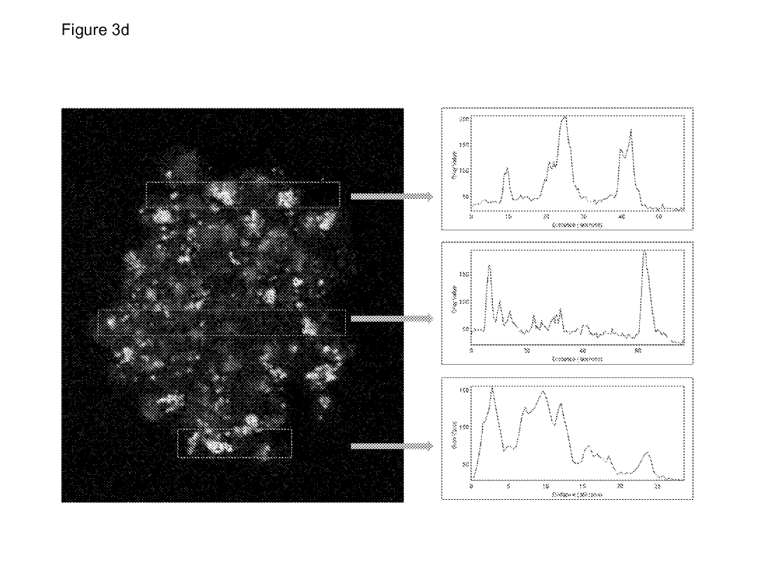

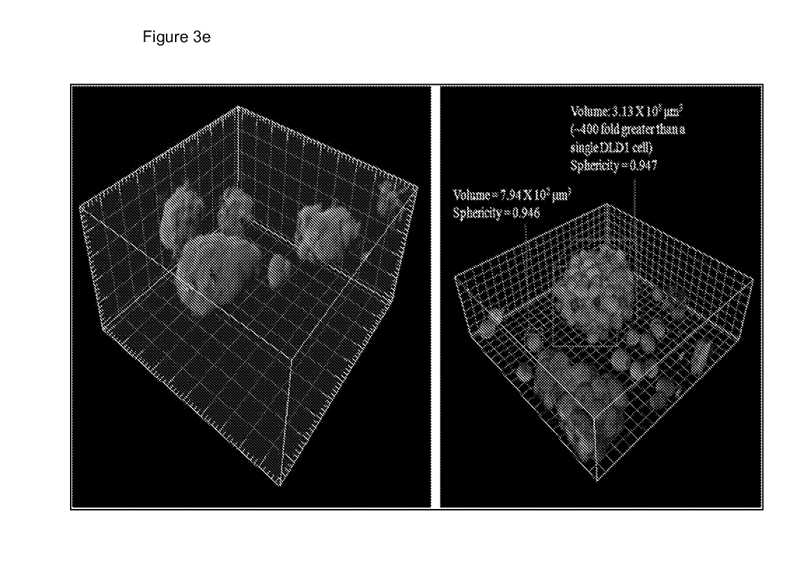

In addition, the microtumors display growth kinetics, active metabolism and mechanical response to the extra cellular matrix that parallels that of a real tumour. In addition, microtumors exhibit prolific levels of acidic components, indicative of active endocytic and secretory mechanisms. These mechanisms allow maintenance of internal homeostasis. FIG. 2, shows the proliferation, in numbers, of microtumours over a 6 day period in per-fused conditions and static conditions. Microtumours exhibit prolific levels of acidic vesicles (FIG. 3a to e) which are prominently present in typical in vivo tumors can be potentially indicative of tumor invasiveness (Montcourrier, Valembois et al. 1993). The release of the contents of acidic vessels into extracellular matrix results in digestion of barriers (i.e., collagen, actin) that normally prevent tumor cell escape (Gatenby and Gawlinski 1996).

According to the present invention, a microtumour is a multi-cellular mass having a diameter of from 30 μm to 150 μm, from 30 μm to 100 μm, from 40 μm to 75 μm, more preferably 50 μm. The relative size of microtumor is about equal in both per-fused and static conditions. It is postulated that the mechanical stress imposed by the surrounding 3D scaffold plays a crucial role in limiting microtumor size.

As used herein, the term “neoplastic cell”, refers to a cell which possesses the capacity for autonomous growth, i.e., capable of rapidly proliferating cellular growth. The term includes cells isolated from all types of cancerous growths or oncogenic processes, metastatic tissues or malignantly transformed cells, tissues, or organs. In one embodiment, the neoplastic cell occurs in disease states characterized by malignant tumour growth. In one embodiment, the neoplastic cell contains a mutation that promotes tumour development and progression.

The term “colorectal neoplastic cell” or “colorectal cancer cell” refers to a cell from a malignancie of the colorectal organ systems. By “colorectal organ systems” is meant the bowels and includes the colon, rectum, small intestine, anus and appendix. The cell may be from an adenocarcinoma, e.g. a colon cancers, or a non-small cell carcinoma, i.e. a cancer of the small intestine. The term “carcinoma” refers to malignancies of epithelial or endocrine tissues, i.e. colon carcinomas. Alternatively, the cell may be from a squamous cell cancer, a carcinoid tumor, a sarcoma or a lymphoma.

In a preferred embodiment, the cell is from the colon. Preferably, the cell is a colon epithelial cell. Preferably, the cell is a colorectal adenocarcinoma. Preferably, the cell is at tumour stage Dukes' type C.

Preferably the cell is a human cell.

The cell may be negative for CSAp (CSAp-). The cell maybe is positive for p53 antigen expression. Preferably, the p53 antigen produced has a C->T mutation resulting in Ser->Phe at position 241. The cell may be positive for keratin by immunoperoxidase staining. The cell may be positive for expression of c-myc, K-ras, H-ras, N-ras, myb, sis and fos oncogenes. The cell may expresses tumour specific nuclear matrix proteins CC-2, CC-3, CC-4, CC-5 and CC-6 are expressed. The cell may include a CHK2 mutation.

In one embodiment the cell is a DLD1 cell, deposited with the ATCC as deposit No. CCL-221. DLD1 contains mutations in genes that regulate cell growth, survival and death leads to promotion of tumor development and progression. Alternatively, the cell is a HCT-15 deposited with the ATCC deposit No. CCL-225.

As used herein, the term “scaffold” refers to any attachment of cells. “Attachment”, “attach” or “attaches” as used herein, refer to cells that adhere directly or indirectly to a substrate as well as cells that adhere to other cells. Preferably, the scaffold is three dimensional i.e. allows cells to grow in more than a single layer. In a preferred embodiment, the scaffold comprises natural polymers.

Preferably, the scaffold is a porous scaffold having an average pore diameter of 75 μm, 100 μm, 150 μm, 200 μm. Preferably, the scaffold is hydrophilic.

Preferably, the scaffold is an alginate based scaffold. As used herein, the term “alginate based scaffold” refers to a scaffold comprising alginate or alginic acid. Alginate is a biodegradable polymer derived from many species, such as algae, i.e. Chlorophyta, Phaeophyta, and Rhodophyta, and from bacteria, such as Azobacter and Pseudomonas.

Alginates are salts of alginic acids. Alginic acids are linear polysaccharides comprising repeating units of D-mannuronic acid (M units) and L-gluronic acid (G units). Alginates may comprise repeating units of D-mannuronic acid (M blocks), repeating units of L-gluronic acid (G blocks) and mixed sequences of D-mannuronic acid and L-gluronic acid (MG blocks).

Preferably, the algenate or algenic acid has a molecular weight of from about 10 kDa to about 1000 kDa, 50 kDa to 500 kDa, more preferably from 200 kDa to 400 kDa, more preferably 200 kDa to 300 kDa, more preferably about 240 to 260 kDa.

Preferably, D-mannuronic acid content is from about 80% to about 50% and L-guluronic acid content is from about 20% to 50%. Preferably, the D-mannuronic acid content is from about 70% to about 55% and L-guluronic acid content is from about 30% to 45%. More preferably, the D-mannuronic acid content is about 61% and the L-guluronic acid content is about 39% respectively, which translates to a M/G ratio of 1.56 (McHugh 1987).

Preferably, the low viscosity alginic acid employed is from the brown algae (Macrocystis pyrifera) or kelp and contains a mixture of D-mannuronic and L-guluronic acids.

Cells seeded into the scaffold in accordance with the present application are grown and passaged using standard cell culture techniques well known in the art. Seeded cells are incubated in a culture medium. Culture media are typically supplemented with animal derived serum products and growth factors as required for specific cell types. In one embodiment, the cells are cultured in serum free culture.

Many nutrient media to support mammalian cell growth are commercially available, such as RPMI-1640, Fischer's medium, Iscoves medium. Microtumor growth may be enhanced by the supplementation of growth factors and regulatory factors to the medium. Preferably, the culture medium is RPMI-1640 supplemented with 10% foetal calf serum. The culture medium may also be supplemented with antibiotics and/or antimycotics well known in the art.

All media components are sterilized, either by heat (20 min at 1.5 bar and 121° C.) or by filter sterilization. The components may be sterilized either together or, if required, separately.

All media components may be present at the start of the cultivation or added continuously or batchwise, as desired. In a preferred embodiment, the cultures are periodically provided with fresh media. Alternatively, the culture may be a perfused system, wherein culture medium continuously flows through the three dimensional scaffold.

In one embodiment the cells are incubated in the scaffold for 2, 3, 4, 5, 6, 7, 8, 9 or 10 days. It is envisaged that the cells may also be incubates for longer or shorter periods of time.

The cell may be incubated at a culture temperature of between 15° C. and 45° C., preferably at from 25° C. to 40° C., more preferably at from 25 to 37° C., more preferably from 35 to 37° C., more preferably at 37° C., and the temperature may be kept constant or may be altered during the experiment.

The pH of the medium should be in the range from 5 to 8.5, preferably around 7.0.

The seeding density should be from about 10¹ cells/ml to 10¹⁰ cells/ml, more preferably from about 10³ cells/ml to 10⁷ cells/ml. More preferably 10⁵ cells/ml are seeded with each scaffold (˜1 mm diameter sphere) containing ˜4×10³ cells.

### Uses

The microtumors of the present application provide an in vitro system for understanding the regulation of colorectal tumour formation and growth.

In addition, the microtumours of the present application provide an in vitro system for evaluating the effectiveness of anti-cancer therapies.

The microtumors of the present application provide an in vitro model for colorectal cancer. Accordingly, the microtumors may be used to identify molecular mechanisms of cancer, in particular colorectal cancers. The microtumors will therefore extend the understanding of cancer and accordingly enable the development of more effective treatment strategies.

In addition, the microtumors may be used to observe the effects of therapeutic agents on cancer, and to identify agents that are capable of treating cancer, in particular colorectal cancer, and reducing the symptoms thereof. The microtumors may be used to identify compounds which have a stimulatory or inhibitor effect upon cancer. The microtumors may be used to identify genetic or chemical agents which modulate (inhibit or delay) cancer progression or which modulate (increase or decrease) tumor sensitivity to radiation or chemical therapy.

Compounds identified by assays described herein may be useful for treating a cancer, in particular a colorectal cancer.

The test compounds used in the methods disclosed herein may, for example, be obtained using any of the combinatorial library methods well known in the art. Such methods include, but are not limited to, those described by Lam, K. S. (1997) Anticancer Drug Des. 12:145.

Example test compounds include, but are not limited to small molecules, peptides, nucleic acids, antibodies, carbohydrates.

The invention provides a method of screening compounds to identify agents useful for treating cancers, in particular colorectal cancers.

The method comprises providing a microtumor formed in accordance with the method described herein, contacting the microtumor with a test compound, and determining the effect of the test compound on the microtumor. For example, the model can be used to determine whether tumor growth or proliferation is inhibited, stimulated, or unchanged.

The test compounds are preferably administered to the microtumors in an amount sufficient to and for a time necessary to exert an effect upon said microtumors. These amounts and times may be determined by the skilled artisan by standard procedures known in the art.

The microtumors of the invention, either within the scaffold or alternatively isolated microtumours in culture, may be exposed to test compounds

The cytotoxic activity of a compound may be assessed by measuring the ability of a compound to kill or damage the cells of the microtumours. Standard staining techniques may be employed to measure cell viability of the microtumours following treatment with a test compound. Vital staining for live/dead cells are well known in the art, for example a LIVE/DEAD® Viability/Cytotoxicity Assay Kit (L-3224) by Molecular Probes, may be used. Such assays identify live versus dead cells on the basis of membrane integrity and esterase activity. Live dead assays may be used to monitor changes in the ratio of live to dead cells in the total cell population. Live dead assays are therefore of particular use in screening compounds which induce cell death. Live dead assays can be used with microscopy, flow cytometry or with a microplate assay. The LIVE/DEAD® Viability/Cytotoxicity Assay Kit (L-3224) uses two fluorescent dyes, calcein AM (cal AM) and ethidium homodimer (EthD-1), to stain live and dead cells simultaneously. Cal AM is an electrically neutral, nonfluorescent, esterase substrate that diffuses into live cells and becomes enzymatically cleaved by ubiquitous cytoplasmic esterases. This releases the free calcein fluorophore that is retained inside live cells. The dye emits a strong green fluorescence that peaks at about 525 nm when excited at about 485 nm. In contrast, EthD-1 is a polar nucleic acid stain that can penetrate dead, but not live cell membranes. Once intercalated into nucleic acids, it produces a 40-fold increase in red fluorescence at about 625 nm when excited at about 525.

The cytostatic properties of a compound may be assessed by measuring the ability of a compound to inhibit the growth of the microtumours and/or proliferation of the cells therein. Alternatively, the cytostatic properties of the compound may be assessed by measuring the microtumour count within the culture or scaffold. The relative size and count of the microtumours from dismantled scaffolds can be readily assessed by flow cytometry based on light scattering. Also, assays such as Alamar Blue and MTT can be readily adapted to quantify the microtumour reduction potential which is a measure of tissue growth and proliferation.

Preferably, the effects of a compound on the cell cycle are assessed. The cell cycle has four phases, G1, S, G2 and M. In G1, a cell grows and differentiates. In S, G2 and M phases the cell propergates itself. Chromosomes are duplicated in S, quality control checked in G2, and equally distributed in two daughter cells in M. Many caner drugs exert their effect on the cell cycle. Therefore quantifying drug effects on various phases of a cell cycle is an ideal measure for assessing a drugs effectiveness. For example a fluorescence activated cell sorter (FACS) based assay or flow cytometery based assay may be used to assess the effect of a test compound on cell cycle.

Alternative markers for monitoring drug effects upon cells are well know in the art and may be used in the methods of the present invention. Two potential intracellular markers for monitoring drug effects on neo-plastic cells and tumors are acidic compartments and endogenous fluorescence emanating from cellular metabolism.

Acidic compartments are mainly endosomes and lysosomes whose interior has a pH of 4. The pH of cytoplasm is between 7 and 8. The maintenance of pH gradients between acidic organelles and neutral/alkaline cytoplasm is essential for endocytotic and secretory mechanisms. These mechanisms maintain internal homeostasis. Defective acidification in organelles results in diminished capacity of cells to remove toxic drugs from the cytoplasm rendering cells more sensitive to drugs, while enhanced acidification is a potential mechanism for drug resistance.

Acidification in organelles is required for many cellular functions including activation of enzymes, packaging of secretory proteins, neutralization of entering pathogens and detoxification of drugs. A disruption of acidification disrupts a cell's homeostatic balance required for survival. One of the mechanisms whereby cancer cells develop resistance to anti neo-plastic drugs is with effective acidic organelles. The quality and quantity of these organelles can server as valuable marker for screening putative anti neo-plastic drugs with a microscopy based technology.

In one example, the acidic compartments can be readily stained with acridine orange (AO). Acridine Orange (AO) is a versatile stain that easily permeates into cells and cellular organelles. AO emits green fluorescence (525 nm) upon intercalating into DNA in the nucleus and red (>630 nm) fluorescence upon protonation in acidic compartments of cytoplasm. This dual emission of AO allows the tracking of cell numbers (via nuclear staining) and their relative acidity (cytoplasm staining). The relative red fluorescence intensity/cell provides a method by which the effectiveness of therapeutic agent may be evaluated in a screening method of the present invention. A therapeutic agent effective against cancerous growth would be expected to decrease the acidity of lysosomal/endocytic vesicles compared to control groups. The red fluorescence generated from within the acidic compartment provide a quality measure of acidity whereas as the green fluorescence from nucleus tracks cell number. The number of acidic granules/cell along with red intensity provide a means by which the effectiveness of a test compound may be determined in a screening method of the present invention.

As shown in FIG. 3 a to e, AO can be used to identify the acidic compartments in DLD1 cells within microtumors. The spatial distribution of acidic organelles is also an important parameter of cell function. It is an active process that is orchestrated by the cell's cytoskeleton. Many anti neo-plastic drugs exert their toxicity by rendering the cytoskeleton useless. This freezes organelle movement leading to cell dysfunction and death. The anti neo-plastic drug paclitaxil (taxol) is known to function in this manner.

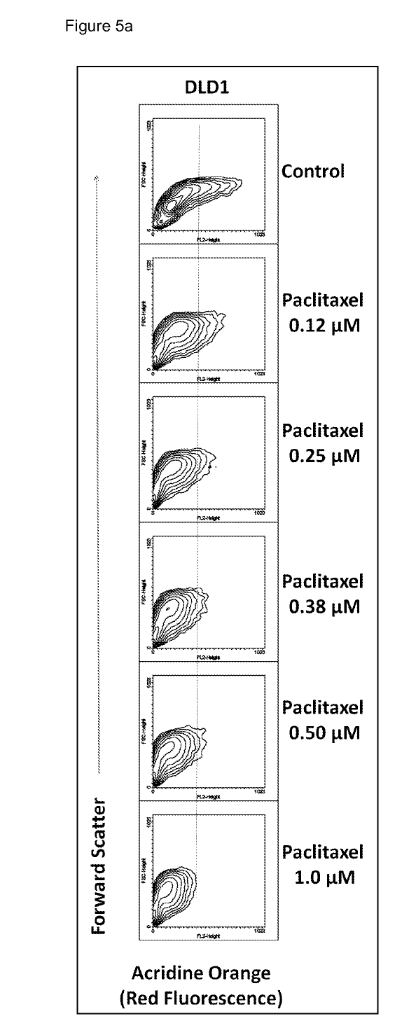

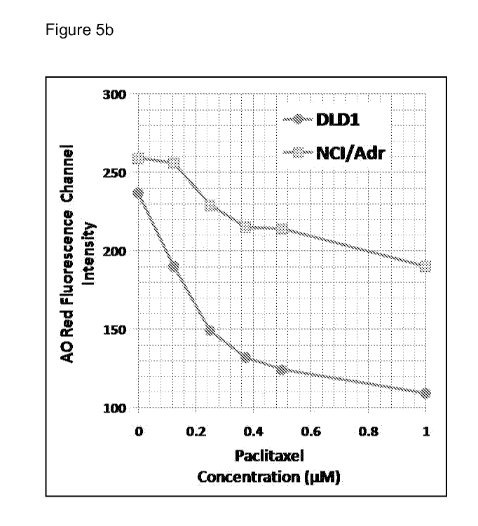

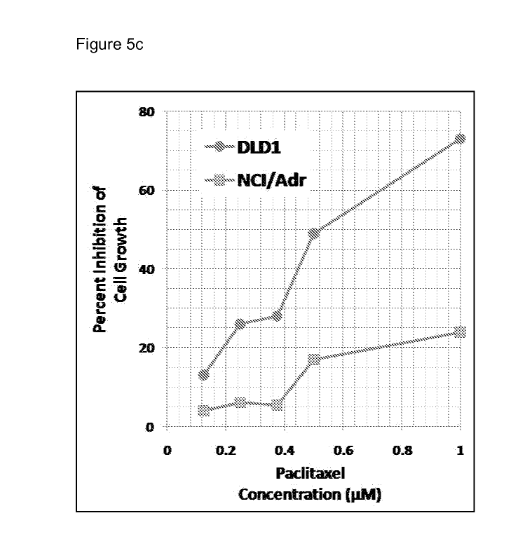

An effective drug against cancer would be expected to lower the pH of acidic compartments and the number of these compartments within a micro-tumor compared to control groups. This is based on the premise that acidification of the tumour micro-environment by acidic compartments provide a chemical barrier for quenching the action of chemotherapeutic drugs (Montcourrier, Mangeat et al. 1994; Gatenby, Gawlinski et al. 2006). FIG. 5 demonstrates that a drug's effectiveness can be assessed with measuring the level of acidity of a cell.

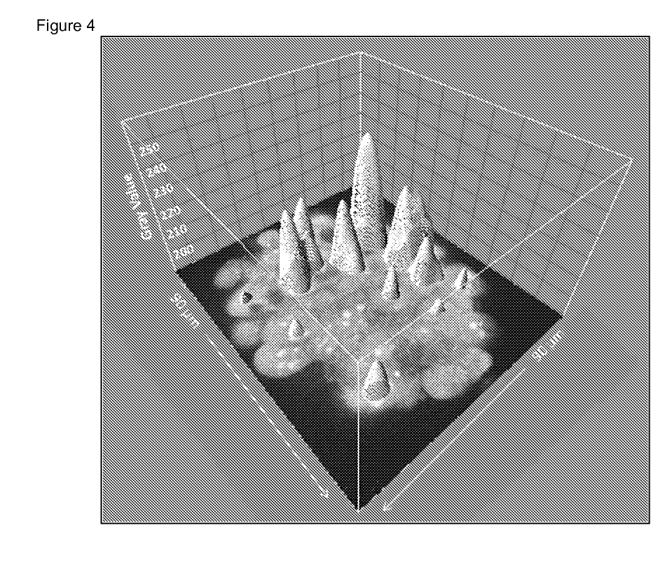

Nuclear staining with acridine orange may also be used to evaluate regions of proliferation, quiescence and necrosis within the microtumors as illustrated in FIG. 4. Proliferative regions contain numerous clusters of dividing nuclei compared to quiescent regions, where singly dispersed nuclei are seen. Necrosis leaves empty pockets where staining with AO is nill.

Endogenous fluorescence (EF) from molecules that orchestrate cellular metabolism (i.e. NAD(P)H and flavoproteins) can be captured by NIR-MPM excitation. Accordingly, these molecules are capable of serving as natural reporters that need not be stained for visualization by NIR-MPM excitation.

Both pyridine nucleotide NAD(P)H and flavoprotein auto fluoresce upon two photon excitation. NAD(P)H is optimally 2-photon excited at wavelengths below 800 nm while flavoproteins are excited with wave lengths above 800 nm. The emission range for both molecules is broad. NAD(P)H: 400-600 nm. Flavoproteins: 450-700 nm (Huang S et al. Biophys J. 2002. 82(5): 2811-2825).

The ability of these molecules to endogenously fluoresce upon NIR-2-photon excitation adds the value of non-invasive monitoring of cell function to microscopy. Both NAD(P)H and flavoproteins are concentrated in the mitochondria and orchestrate cellular respiration, so therefore the fluorescence intensity of these molecules closely correlate with cellular metabolism. These molecules may therefore be used to qualitatively and quantitatively measure a cell's energy state.

The assessment of a cell's energy state can be a viable option for screening anti neo-plastic drugs. Drugs can take their effect on a number of places inside the cell including mitochondria. Drugs that can induce mitochondrial outer membrane permeabilization (MOMP) are viable candidates. MOMP leads to apoptotic death. The inability of cancers to respond to apoptotic inducing drugs determines their aggressive nature—the classic example of this being pancreatic cancer.

In addition, cancer cells also synthesize ATP through aerobic glycolysis resulting in elevated NAD(P)H and glutathione—both increases the resistance of cancer cells to oxidative damage and certain cancer drugs.

Accordingly, the Endogenous fluorescence (EF) of NAD(P)H and flavoproteins provides a means by which the effectiveness of a test compound may be determined in a screening method of the present invention. Since cellular metabolism is indicative of cell growth, drugs of cytotoxic nature including anti-neoplastic drugs would be expected to quench auto-fluorescence emanating from NAD(P)H and flavorproteins.

In addition, non-invasive imaging of cellular endogenous fluorescence can be used to for real-time monitoring of drug effects on microtumors of the present invention.

### EXAMPLES

Materials were purchased from Sigma-Aldrich Chemical Company (Dorset, UK) unless when indicated.

DLD1 cells (human adenoma colorectal cancer cell line): Cells were obtained from Richard Callaghan, Nuffield Department of Clinical Laboratory, John Radcliffe Hospital, University of Oxford, UK).

Growth media. RPMI-140 medium modified—without phenol red and sodium bicarbonate. Prior to use, the media was supplemented with 25 mM HEPES buffer, 10% heat-inactivated fetal calf sera and antibiotic-antimycotic solution (100 units/ml penicillin G, 0.01 mg/ml streptomycin sulfate and 0.25 μg/ml amphotericin B).

Tissue disaggregation: Trypsin-EDTA; porcine trypsin (0.5 g/l) and EDTA (0.2 g/l). Dispase (10 U/ml) —purchased from Gibco/InVitrogen, Paisley, UK.

Alginate scaffold. Low viscosity alginate (Merck, UK), 1.2% (w/v) in a 0.9% NaCl solution. Cross linking agent, 102 mM CaCl₂. De-cross linking agent, Na-Citrate buffer.

Microscopy staining: Acridine Orange (3,6-Bis (dimethyl.amino, acridine. hydrochloride).

Ethidium Bromide. Trypan Blue (0.4% w/v). ANS (8-Anilino-1-naphthalene Sulfonic Acid) —purchased from Molecular Probes/InVitrogen.

### Preparation of Monolayers

Monolayer cultures and preparation of single cell suspensions: Stock DLD1 cells (human adenoma colorectal cancer cell line) were seeded at a density of 1.5×10⁵ cells/cm² in a 75 cm² tissue culture flask. Growth media was added and incubated at 37° C. in humid conditions until cells reached 50% confluences—mid log phase growth. For single cell suspension, monolayers (after the growth media was removed) were detached from the tissue culture with trypsin-EDTA (10 ml, 15 minutes at 37° C.), transferred to a 50 ml conical centrifuge tube and mechanically agitated (repeated pipetting) to dislodge cells from each other. Cells were washed by adding 30 ml of growth media to the suspension and centrifuged at 100×g for 10 min at room temperature. Washing was repeated 2 more times. Cell viability and enumeration was determine by trypan blue exclusion method. Viability was greater than 97%.

### Preparation of Scaffolds

Generation of micro-tumors in alginate scaffold. The method employed to encapsulate cells into alginate scaffolds is described elsewhere (Xu, Urban et al. Osteoarthritis Cartilage 15(4): 396-402. 2007). Briefly mono-dispersed DLD1 cells were gently mixed with alginate solution to final concentrations of 10⁵ cells/ml and 1% alginate. The cell/alginate mix was then transferred into a 5 cc syringe and drop-wise delivered through a 22 gauge needle into a solution of CaCl₂ (102 mM). Upon drop-wise delivery, the alginate polymers immediately begin to cross-link forming spherical beds in the solution. After an additional 10 minutes of incubation in the CaCl₂ solution to fully ensure the cross-linking process, the beds were washed thrice in 0.9% NaCl solution (room temperature, 30 min) and one in growth media (37° C., overnight). The following day, alginate beds were transferred to either a 96-well formatted parallel micro-reactor for per-fusion based culturing or 24-well plates for static culturing for various time points, with 6 day being the maximum.

### Microtumor Visualization

Imaging by Near-Infrared Microscopy. At various time beds were removed from culture and stained for microscopy. Beds were either stained with acridine orange (C_{f}=100 μg/ml), ANS (C_{f}=1 mM) or AO/EB cocktail (C_{f}=100 μg/ml, 400 μg/ml). Imaging was conducted with ˜800 nm pulsed laser light visualized with 60× or 10× objective lens.

Alginate beds were stained for live/dead cell discrimination with a cocktail of AO and ethidium bromide (EB). Like AO, EB intercalates into DNA and fluoresces red. However, EB only is able penetrate into dead cells. The differential entry of dyes and fluorescence emission provide a litmus-measure for cell viability.

As illustrated in FIG. 1 DLD1 cells were seeded into scaffolds as mono-dispersed cells (day 0) and by day 6 both static and per-fused cultures show ample numbers of microtumors. The images of FIG. 1 are raw gray scale recordings of fluorescence emanating from acridine orange (AO) bound to DNA within cells upon 2-photon excitation by a near infrared-multiphoton laser scanning microscope (NIR-MPLSM). The excitation wave length employed was ˜800 nm with the ensuing green emission of AO (˜525 nm) being visualized through a 10× water immersion objective lens and a 500-530 nm emission filter. In images not shown here, the scaffolds also stained with ethidium bromide (EB) exhibited little or no fluorescence in the emission range of 607-682 nm was observed. Like AO, EB also binds to DNA but emits red light (˜610 nm) and penetrates into only dead cells demonstrating viability of cells and microtumors contained within the scaffold. Images were processed for documentation with ImageJ, a public domain software made available by the National Institutes of Health (http://rsbweb.nih.gov/ij/).

### Quantifying Size and Number of Microtumors in 3D Alginate Cultures

The NIR-MPLSM laser system used in this study consists of a diode pumped Ti:Sapphire crystal laser (Mira-Coherent, Ely, UK) coupled to a BioRad Radiance 2100 mulitphoton dedicated laser-scanning system (CarlZeiss Hertforshire, UK) and a Nikon E600 FN upright microscope (Nikon UK Ltd, Surrey, UK). The 10 W solid sate pump laser and Ti:Sapphire crystal laser (Coherent, Ely, UK) provides 150 femto-second pulses of NIR light that is tunable between 700 and 980 nm. Images were acquired with LaserSharp software (Carl Zeiss, Hertforshire, UK) and post processing/analysed with either ImageJ or IMARIS (Bitplane Ag, Zurich, Switzerland) software programs.

A multi-cellular mass of ≧50 μm in diameter was designated as a microtumor. Mono-dispersed DLD1 cells in the same culturing conditions have a diameter of 10.8 μm (+1.3, n=6).

The diameter of each MT was determined using IMARIS software (Bitplane Ag, Zurich, Switzerland). IMARIS software is a three-dimensional imaging tool that includes volume rendering, orthogonal plane projections and statistical analysis of three-dimensional objects that includes—size—volume—and fluorescence intensity assessment. Prior to generating statistics, the images were adjusting, via threshold setting to quench or remove background noise in the pertinent channel (AO or green fluorescence). The results of these measurements are provided in Table 1 below, which provides an assessment of micro-tumour size in alginate scaffolds.

TABLE 1
Growth of Micro-tumor size (diameter in micrometers)

| | Static culture | Per-fused culture |
|---|---|---|
| Day 4 | 57, n = 1 | — |
| Day 5 | 64 ± 13, n = 7 | 62 μm ± 9, n = 8 |
| Day 6 | 66 ± 16, n = 19 | 64 μm ± 12, n = 31 |

As described for table 1 above, the total numbers of microtumors in a 1.2 mm×1.2 mm image were generated by Imaris software. Prior to generating statistics, threshold for green fluorescence channel (AO fluorescence) was triggered to quantify objects ≧50 μm in diameter. The proliferation of the microtumors in alginate scaffolds either in a static or per-fused culture is illustrated in FIG. 2.

### Image Analysis of Micro-Tumors in 3-D Alginate Cultures

Microtumor size and numbers were quantified from NIR-MPM images using Imaris 4.5 (Bitplane Ag, Zurich, Switzerland) software using the measurement pro-module. The measurement-pro module allows enumerating objects within an image from one the three channels recorded (blue, green or red fluorescence). In this case, by modulating the threshold setting for green fluorescence, objects greater than 50 μm within the image were selected—based on this setting, the software automatically compiles a number of statistical measures of the objects including their total number within a given image.

FIG. 3a illustrates that dual fluorescence of AO reveals both nuclear and acidic components within a microtumor. AO emits green fluorescence (˜525 nm) upon binding to DNA (i.e., nucleus) and red fluorescence (>630 nm) when protonated in acidic components (i.e., lysosomes). A microtumor in alginate scaffold was stained with AO and visualized by a multi-photon microscope with a 60× water objective lens and 800 nm excitation. The above gray scale images are that of an optical section (10 μm below the MT surface) showing fluorescence collected in the 3 emission channels. AO has no known fluorescence in the blue (435-485 nm). Fluorescence collected between 500-535 nm is that of nuclear staining and residual dye. Fluorescence collected between 607-682 nm is that acidic components. Images were processed for documentation with ImageJ.

FIG. 3b provides a cross-sectional map of the acidic components of a microtumor grown under per-fused culture conditions (day 6). Optical sections (37 in total) collected from imaging an entire microtumor were collapsed into a single inverted image—the lighter shades of a gray scale recording (˜higher fluorescence intensities of AO fluorescence) become darker shades of gray. In the green emission channel, the darker shades are the nuclei and in the red emission channel are the acidic components. As the figure illustrates, geography of green and red fluorescence from within the microtumor is distinct.

FIG. 3c illustrates the Acidic components distributed across a Microtumor at various depths from the surface. These images are from the sample employed in FIGS. 3 and 4 where red fluorescence generated by AO was captured in the emission range between 607-682 nm. Images were constructed using ImageJ software.

FIG. 3d illustrates the relative acidity of acidic components distributed across a microtumor. Lower the pH, the higher the intensity of AO red fluorescence. In this figure are line scale analysis (dotted boxes) extracting gray scale intensity values of AO red fluorescence from three regions at 20 μm depth within a microtumor. In cancer, acidic components play a crucial in helping establish an acidic microenvironment around a tumor which aids it tumor progression.

FIG. 3e illustrates the 3D constructs of microtumor grown in alginate scaffolds. Scaffolds were stained with AO and imaged with ˜800 nm light with a 60× water objective lens. The collected optical sections were 3D volume rendered using Imaris software (Bitplane Ag, Zurich, Switzerland).

### Identification of Proliferative Regions

FIG. 4 identifies proliferative regions within a microtumor. Proliferative (P), quiescent (Q) and necrotic regions (N) within a microtumor can be identified based on the relative intensity of green fluorescence emanating from AO-DNA complex. P regions are characterized by dense clusters of dividing nuclei and Q regions by mono-dispersed non-dividing nuclei. Necrotic regions due to cellular degradation would contain no nuclei. So, the relative intensity of fluorescence of AO for the regions would be P>Q>N. In the above 3D surface plot, an optical section 40 μm below the surface of the microtumor was mapped (using ImageJ) for P regions represented as histograms (z-axis, gray scale units) protruding from the base image. The histograms were generated by setting a lower limit of 200 gray scale units (AO green fluorescence channel) in the software module (ImageJ) to drown out Q regions—Q regions are mono-dispersed cells within image and have a fluorescence intensity ≦200 gray scale units. Dark pockets that appear in the image are most likely N regions. Initially, the image was acquired with a 60× water objective lens and 800 nm of NIR-pulsed laser light.

### Microtumor Dispersion

Attempts to disperse microtumors into single cell suspensions for flow-cytometric analysis. Micro-tumours formed on day 5 and 6 were extracted by incubating alginate beds in Na-Citrate buffer (15 minutes at room temperature). Beds were then washed twice in PBS/EDTA and incubated in the following digestives:
i) Trypsin-EDTA. Beds were treated either for 15 minutes or 2 hours (with mechanical agitation) at 37° C. The 2-hour treatment included a mechanical agitation step every 30 min.
ii) Dispase treatment. Dispase is a metalloprotease of Bacillus origin (Stenn, Link et al. J Invest Dermatol 93(2): 287-90. 1989). With dispase treatment, single cell suspension from were obtained readily (15-20 min) from the skin's epidermis (Kitano and Okada Br J Dermatol 108(5): 555-60. 1983) and tissue aggregates in Matrigel™ (Strick-Marchand and Weiss Hepatology 36(4 Pt 1): 794-804. 2002). In the same manner as trypsin-EDTA treatment, gel beds were incubated in 1 U/ml dispase for 15 minutes or 2 hours (with mechanical agitation) at 37° C.

Attempts to disperse the microtumors into a single cell suspension were unsuccessful. Microtumors remained intact as assessed by microscopic/hemacytometer examination. Although some cell debris were observed, monodispersed cells were not obtained.

### Effect of Anti-Cancer Drugs on Cellular Acidity

As illustrated in FIG. 5, the acidic content (˜red fluorescence of AO) of cancer cells was correlated with cell growth. Cells plated at 25% confluency were exposed to various doses of the drug for 6 hours and stained with AO (1 μg/ml, final concentration) for 30 minutes. Cells were washed 3× with PBS to remove residual stain, trypsinized to obtain a liquid suspension and assessed on a flow cytometer (FACSCalibur, Becton Dickinson San Jose Calif.). The red fluorescence of AO emanating from the cells was recorded on a linear scale in the cytometer's FL2 channel (emission filter >600 nm). A total of 30K cells were collected for post acquisition analysis with WinMDI Version 2.8 software (The Scripps Research Institute, La Jolla, Calif.). FIG. 5a shows 10% contour diagrams plotting forward scatter (FSC) vs AO red fluorescence channel intensity of DLD1 cells exposed to various drug doses. FIG. 5b plots the geometric mean of AO red fluorescence channel intensity against drug concentration for both DLD1 and NCl/Adr cells. In parallel cultures, cells were assessed for growth 3 days after the addition of drug (FIG. 5 c) —cells were enumerated with a hemacytometer and trypan blue exclusion staining. As the results indicate, at a given dose DLD1 cells are more sensitive to paclitaxel than NCl/Adr and this sensitivity can reflected by the acidic content of cells.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

Burleson, K. M., M. P. Boente, et al. (2006). “Disaggregation and invasion of ovarian carcinoma ascites spheroids.” J Transl Med 4: 6.
Chatterjee, S. S. and M. Noldner (1994). “An aggregate brain cell culture model for studying neuronal degeneration and regeneration.” J Neural Transm Suppl 44: 47-60.
Dierssen, J. W., N. F. de Miranda, et al. (2006). “High-resolution analysis of HLA class I alterations in colorectal cancer.” BMC Cancer 6: 233.
Dvorak, H. F. (1986). “Tumors: wounds that do not heal. Similarities between tumor stroma generation and wound healing.” N Enql J Med 315(26): 1650-9.
Dvorak, H. F., J. A. Nagy, et al. (1991). “Structure of solid tumors and their vasculature: implications for therapy with monoclonal antibodies.” Cancer Cells 3(3): 77-85.
Franko, A. J. (1985). “Hypoxic fraction and binding of misonidazole in EMT6/Ed multicellular tumor spheroids.” Radiat Res 103(1): 89-97.
Freyer, J. P. and R. M. Sutherland (1986). “Regulation of growth saturation and development of necrosis in EMT6/Ro multicellular spheroids by the glucose and oxygen supply.” Cancer Res 46(7): 3504-12.
Gatenby, R. A. and E. T. Gawlinski (1996). “A reaction-diffusion model of cancer invasion.” Cancer Res 56(24): 5745-53.
Gillies, R. J., N. Raghunand, et al. (2002). “MRI of the tumor microenvironment.” J Maqn Reson Imaging 16(4): 430-50.
Kaplan, D. L. and W. F. Boron (1994). “Long-term expression of c-H-ras stimulates Na—H and Na(+)-dependent Cl-HCO3 exchange in NIH-3T3 fibroblasts.” J Biol Chem 269(6): 4116-24.
Khaitan, D., S. Chandna, et al. (2006). “Establishment and characterization of multicellular spheroids from a human glioma cell line; Implications for tumor therapy.” J Transl Med 4: 12.
Kitano, Y. and N. Okada (1983). “Separation of the epidermal sheet by dispase.” Br J Dermatol 108(5): 555-60.
Kurosawa, H., T. Imamura, et al. (2003). “A simple method for forming embryoid body from mouse embryonic stem cells.” J Biosci Bioeng 96(4): 409-11.
Landry, J., E. Lord, et al. (1982). “In Vivo Growth of Tumor Cell Spheroids after in Vitro Hyperthermia.” Cancer Research 42: 6.
Mellor, H. R., D. J. Ferguson, et al. (2005). “A model of quiescent tumour microregions for evaluating multicellular resistance to chemotherapeutic drugs.” Br J Cancer 93(3): 302-9.
Montcourrier, P., P. H. Mangeat, et al. (1994). “Characterization of very acidic phagosomes in breast cancer cells and their association with invasion.” J Cell Sci 107 (Pt 9): 2381-91.
Montcourrier, P., I. Silver, et al. (1997). “Breast cancer cells have a high capacity to acidify extracellular milieu by a dual mechanism.” Clin Exp Metastasis 15(4): 382-92.
Montcourrier, P., C. Valembois, et al. (1993). “[The presence of large acidic vesicles is correlated with in vitro invasive potential of breast cancer cells].” C R Acad Sci III 316(4):421-4.
Mueller-Klieser, W. (1987). “Multicellular spheroids. A review on cellular aggregates in cancer research.” J Cancer Res Clin Oncol 113(2): 101-22.
Nicholson, K. M., M. C. Bibby, et al. (1997). “Influence of drug exposure parameters on the activity of paclitaxel in multicellular spheroids.” Eur J Cancer 33(8): 1291-8.
Parkins, C. S., M. R. Stratford, et al. (1997). “The relationship between extracellular lactate and tumour pH in a murine tumour model of ischaemia-reperfusion.” Br J Cancer 75(3): 319-23.
Postovit, L. M., E. A. Seftor, et al. (2006). “A three-dimensional model to study the epigenetic effects induced by the microenvironment of human embryonic stem cells.” Stem Cells 24(3): 501-5.
Santini, M. T. and G. Rainaldi (1999). “Three-dimensional spheroid model in tumor biology.” Pathobiology 67(3): 148-57.
Sharma, M., Y. Verma, et al. (2006). “Imaging growth dynamics of tumour spheroids using optical coherence tomography” Biotechnology Letters 29(2): 5.
Stenn, K. S., R. Link, et al. (1989). “Dispase, a neutral protease from Bacillus polymyxa, is a powerful fibronectinase and type IV collagenase.” J Invest Dermatol 93(2): 287-90.
Strick-Marchand, H. and M. C. Weiss (2002). “Inducible differentiation and morphogenesis of bipotential liver cell lines from wild-type mouse embryos.” Hepatology 36(4 Pt 1): 794-804.
Sutherland, R. M. (1988). “Cell and environment interactions in tumor microregions: the multicell spheroid model.” Science 240(4849): 177-84.
Wu, F. J., J. R. Friend, et al. (1999). “Enhanced cytochrome P450 IA1 activity of self-assembled rat hepatocyte spheroids.” Cell Transplant 8(3): 233-46.
Xu, L., D. Fukumura, et al. (2002). “Acidic extracellular pH induces vascular endothelial growth factor (VEGF) in human glioblastoma cells via ERK1/2 MAPK signaling pathway: mechanism of low pH-induced VEGF.” J Biol Chem 277(13): 11368-74.
Xu, X., J. P. Urban, et al. (2007). “Influences of buffer systems on chondrocyte growth during long-term culture in alginate.” Osteoarthritis Cartilage 15(4): 396-402.
Gatenby, R. A., E. T. Gawlinski, et al. (2006). “Acid-mediated tumor invasion: a multidisciplinary study.” Cancer Res 66(10): 5216-23.
McHugh, D. J., Ed. (1987). Production and utilization of products from commercial seaweeds FAO Fish. Tech. Pap.
Montcourrier, P., P. H. Mangeat, et al. (1994). “Characterization of very acidic phagosomes in breast cancer cells and their association with invasion.” J Cell Sci 107 (Pt 9): 2381-91.

1. A method of producing an in vitro microtumour comprising:
a) seeding a colorectal neoplastic cell into a three dimensional scaffold comprising polysaccharide co-polymer;
b) providing said cell with a culture medium that supports the growth thereof; and
c) incubating said cell in said scaffold for a time sufficient for microtumors to form,
wherein said polysaccharide co-polymer comprises glutaronate and mannuronate.

2. The method of claim 1, wherein said cell is a colorectal adenocarcinoma cell.

3. The method of claim 1, wherein said cell comprises a CHK2 mutation.

4. The method of claim 1, wherein said cell is a colon cell.

5. The method of claim 1, wherein said cell is a DLD1 cell.

6. The method of claim 1, wherein said culture medium is provided as a static culture.

7. The method of claim 1, wherein said culture medium is provided as a perfused culture.

8. The method of claim 1, wherein said polysaccharide is an alginate or alginic acid.

9. A method of screening a compound to identify agents useful for the treatment of cancer, comprising:
a) exposing a microtumor to a test compound; and
b) determining the effect of the compound on the microtumor,
wherein said microtumor is produced by seeding a colorectal neoplastic cell into a three dimensional scaffold which comprises a polysaccharide co-polymer comprising glutaronate and mannuronate.

10. The method of claim 9, wherein said microtumor is within said three dimensional scaffold.

11. The method of claim 9, wherein said cell is a colorectal adenocarcinoma cell.

12. The method of claim 9, wherein said cell comprises a CHK2 mutation.

13. The method of claim 9, wherein said cell is a colon cell.

14. The method of claim 9, wherein said cell is a DLD1 cell.

15. The method of claim 9, wherein the method further comprises determining the cytotoxic effect of the test compound on the microtumor.

16. The method of claim 15, wherein the cytotoxic effect of the test compound on the microtumor is determined using a live/dead cytotoxicity assay.

17. The method of claim 9, wherein the method further comprises determining the effect of the test compound on acidic compartments of the microtumor.

18. The method of claim 17, wherein the effect of the test compound on acidic compartments of the microtumor is determined by staining of the microtumor with acridine orange.

19. The method of claim 9, wherein the method further comprises determining the effect of the test compound on endogenous fluorescence of the microtumor.

20. The method of claim 19, wherein the effect of the test compound on endogenous florescence is determined by the visualisation of luroproteins by NIR-MPM.

21. The method of claim 9, wherein the method further comprises determining the cytostatic effect of the test compound on the microtumor.

22. The method of claim 9, wherein said polysaccharide co-polymer comprising glutaronate and mannuronate is alginate or algenic acid.

23. A process for preparing a pharmaceutical composition for treating a colorectal cancer comprising:
(a) screening a plurality of compounds using a microtumor produced by seeding a colorectal neoplastic cell into a three dimensional scaffold comprising glutaronate and mannuronate to determine the effect of the compound on the microtumor;
(b) selecting from the plurality a compound having a cytotoxic or cytostatic action against said microtumor;
(c) synthesising the selected compound; and
(d) incorporating the synthesized compound into a pharmaceutical composition.

24. (canceled)

25. A microtumor produced by the method of claim 1.

26. A three dimensional alginate scaffold comprising a colorectal neoplastic cell aggregate which is resistant to trypsin digestion.

27.-29. (canceled)
